(19) United States
(12) Patent Application Publication
(10) Pub. No.: US 2026/0114927 A1
(43) Pub. Date: Apr. 30, 2026

(54) APPARATUS FOR POSITIONING A MEDICAL OBJECT AND METHOD FOR EMITTING A LIGHT DISTRIBUTION

(71) Applicant: Siemens Healthineers AG, Forchheim (DE)

(72) Inventor: Alois Regensburger, Poxdorf (DE)

(21) Appl. No.: 19/375,198

(22) Filed: Oct. 30, 2025

(30) Foreign Application Priority Data
Oct. 31, 2024 (DE) 10 2024 210 524.4
Nov. 4, 2024 (DE) 10 2024 210 563.5

(51) Int. Cl. A61B 34/10; A61B 17/34; A61B 90/00; A61B 90/30

(52) U.S. Cl. CPC A61B 34/10; A61B 17/3403; A61B 90/06; A61B 90/08; A61B 90/30; A61B 90/37; A61B 90/39; A61B 2034/107; A61B 2090/0807; A61B 2090/376; A61B 2090/3937

(57) ABSTRACT

An apparatus for positioning a medical object includes a control unit and a light guidance unit. The control unit is configured to provide a planning information item regarding a planned positioning of the medical object. The light guidance unit is configured to emit at least one light fan beam dependent upon the planning information. The at least one light fan beam has two different angle regions that abut one another along a boundary line. The two different angle regions and/or the boundary line has a visually distinguishable property. The boundary line illuminates at least a part of the planned positioning of the medical object.

This application claims the benefit of German Patent Application No. DE 10 2024 210 524.4, filed on Oct. 31, 2024, and German Patent Application No. DE 10 2024 210 563.5, filed on Nov. 4, 2024, which are hereby incorporated by reference in its entirety.

### BACKGROUND

The present embodiments relate to an apparatus for positioning a medical object, a method for emitting a light distribution, and a computer program product.

During medical interventions, a precise placement of medical objects (e.g., a needle and/or a medical instrument) may be of decisive significance (e.g., during minimally invasive interventions, such as biopsies, pain therapy and/or the placement of catheters). The medical object may be introduced along a planned trajectory to a target location and/or oriented along a planned trajectory (e.g., a planned spatial direction). For example, in the case of a vertebroplasty, a method for stabilizing vertebral bodies, as well as in other percutaneous interventions (e.g., an intervention on a hip bone) and/or lung procedures (e.g., an ablation), an exact placement of the needles may play a central role. Often, laser needle guidance systems that are integrated into a detector of a C-arm X-ray device are made use of. However, these systems function only under particular conditions (e.g., if a planned needle path is in a defined position relative to the detector). A common problem in the use of the existing laser needle guidance systems is their limited flexibility. During vertebroplasty, the laser needle guidance may only be used for a bull's-eye view, whereas, for practical reasons, the progression views that are oriented perpendicular to the needle path are insufficient for medical personnel. In addition, in many percutaneous hip bone procedures, the bull's-eye view and sometimes also the progression view are not readily accessible due to collision problems. Setting the correct trajectory of the needle is therefore often difficult and time-consuming. In lung procedures, a 3D trajectory guidance and lateral fluoroscopy in the progression view would be extremely helpful. Currently, however, a laser on a C-arm X-ray device does not offer complete trajectory information in the progression view.

In order to solve these problems, various approaches have previously been followed. For example, conventional laser needle guidance may be used on a C-arm X-ray device, or the procedure may be carried out with a computed tomography (CT) device in place of the C-arm X-ray device. Laser needle guidance systems that are based upon tiltable mirrors may be utilized. Disadvantageously, these mechanically complex laser needle guidance systems require more structural space and are more expensive. Alternatively, external navigation systems may be used. Alternatively, guidance systems may be dispensed with entirely, and it is possible to work with just fluoroscopic imaging.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an improved positioning of medical objects is provided. Independent of the grammatical term usage, individuals with male, female, or other gender identities are included within the term.

The present embodiments relate, in a first aspect, to an apparatus for positioning a medical object. The apparatus includes a control unit (e.g., a controller including one or more processors) and a light guidance unit. The control unit is configured to provide planning information regarding a planned positioning of the medical object. The light guidance unit is configured to emit at least one light fan beam dependent upon the planning information. The at least one light fan beam has two different angle regions that abut one another along a boundary line. The angle regions and/or the boundary line have a visually distinguishable property. In an operating state of the apparatus, the boundary line illuminates at least a part of the planned positioning of the medical object.

The medical object may include a surgical instrument (e.g., a needle, such as a puncture needle and/or a drill and/or a trocar, such as a bone trocar, and/or a diagnostic instrument, such as an endoscope, such as a laparoscope, and/or a catheter and/or a guide wire, such as a K-wire, and/or an implant). In one embodiment, the medical object may be configured at least partially (e.g., completely) rigid and elongate (e.g., rod-shaped and/or needle-shaped).

The provision of the planning information may include, for example, an acquisition and/or a readout of a computer-readable data store and/or a reception from a data storage unit (e.g., a database). Additionally, the planning information may be provided by a provisioning unit of a medical imaging device. Additionally, the control unit may be configured to provide the planning information to the light guidance unit (e.g., using a corresponding signal).

The planning information may have an information item (e.g., a specification) regarding a planned spatial positioning (e.g., a spatial position and/or orientation and/or attitude and/or trajectory) of the medical object. Therein, the planning information may specify the planned positioning of the medical object (e.g., in relation to the examination object, such as in relation to a coordinate system of an examination object). The examination object may be, for example, a human and/or animal patient and/or an examination phantom.

The light guidance facility may include a light source (e.g., a laser light source) that is configured to emit the at least one light fan beam. For this purpose, the light guidance facility may include, for example, an optical aperture. The at least one light fan beam may illuminate a pre-defined slice (e.g., in a fan form).

The at least one light fan beam has two different angle regions that abut one another along a boundary line. Further, the at least one light fan beam may have further angle regions that abut one another in pairs along a boundary line in each case. The at least one light fan beam may be capable of being emitted fan-shaped within an overall angle region with the light source as the apex of the light guidance unit. The overall angle region may include the at least two angle regions (e.g., may be formed from the at least two angle regions). Therein, the at least one boundary line may extend between the at least two angle regions through the apex of the overall angle region. For example, the boundary line may extend along a common limb of the angle regions which abut one another. The at least two angle regions may thus have the common apex point. The at least two angle regions may each be defined by an angle in the common apex point, where a sum of the angles of the at least two angle regions corresponds to an angle of the overall angle region in the apex. The respective angles of the at least two angle regions in the common apex point may be identical or different.

The at least two angle regions may have a visually distinguishable property. For example, due to their visually distinguishable property, the at least two angle regions may be distinguishable (e.g., identifiable). Alternatively or additionally, the boundary line may have a visually distinguishable property. Therein, the boundary line may be distinguishable from the at least two angle regions due to its visually distinguishable property. According to one embodiment, both the at least two angle regions and also the boundary line may each be distinguishable (e.g., identifiable) due to the respective visually distinguishable properties.

The boundary line may project a boundary region (e.g., a boundary point or a transition point) that is formed on an intersection region or on a border of the lines projected via the at least two angle regions of the light fan beam. The boundary point may relate to a point projected onto a surface, at which the two angle regions of the light fan beam have (e.g., in a visual perception by a human eye) a defined (e.g., identical or weighted) brightness and/or intensity. The boundary region may also denote a region on the border or intersection of the mutually abutting angle regions of the light fan beam, in which the human eye perceives a particular mixed color from light colors (e.g., laser colors) of the respective angle regions.

The light guidance facility is configured to emit the at least one light fan beam dependent upon the planning information such that the boundary line illuminates at least a part of the planned positioning of the medical object. According to one embodiment, the boundary line may illuminate a planned path for positioning the medical object. According to a further embodiment, the boundary line may illuminate a predefined region (e.g., a marker structure) on the medical object and/or a further object (e.g., the examination object) if the medical object is positioned in accordance with the planned positioning.

The apparatus of the present embodiments may enable an improved positioning of the medical object.

In a further embodiment of the apparatus, the visually distinguishable property of the angle regions and/or of the boundary line may include at least one of the following features: a color, a spatial and/or temporal pattern, a polarization, a brightness, a line thickness, and/or an unsharpness.

In one embodiment, the visually distinguishable property of the at least two angle regions and/or of the boundary line may include a color (e.g., a light wavelength and/or a light wavelength distribution and/or a light wavelength mixture). In one embodiment, the respective color may be detectable by a human observer. If the at least two angle regions have a different color, then the boundary line may be formed by a color transition between the two angle regions.

Alternatively or additionally, the visually distinguishable property of the at least two angle regions and/or of the boundary line may include a spatial and/or temporal pattern. The spatial pattern may be formed, for example, by a spatial contrast distribution and/or a spatial color distribution and/or a spatial brightness distribution. The temporal pattern may be formed, for example, by a sequence (e.g., a temporal sequence) of a predefined light distribution that is different for the at least two angle regions and/or the boundary line.

In one embodiment, the visually distinguishable property of the at least two angle regions and/or of the boundary line may include a polarization. In one embodiment, the respective polarization may be detectable by a human observer (e.g., using a pair of polarization glasses and/or a polarization-selective reflection).

Alternatively or additionally, the visually distinguishable property of the at least two angle regions and/or of the boundary line may include a brightness difference.

Alternatively or additionally, the visually distinguishable property of the at least two angle regions and/or of the boundary line may include a line thickness. In one embodiment, the at least one light fan beam may project a light pattern onto a surface (e.g., the medical object and/or a further object, such as the examination object). Therein, the at least two angle regions and/or the boundary line may have a different line thickness (e.g., a different width) of each projected line.

Alternatively or additionally, the visually distinguishable property of the at least two angle regions and/or of the boundary line may include an unsharpness. Therein, the at least two angle regions and/or the boundary line may have a different unsharpness (e.g., in each edge region of the projected light pattern).

The embodiment may enable a visual detection of the at least two angle regions and/or of the boundary line by a human observer (e.g., a medical operating person).

In a further embodiment of the apparatus, the light guidance unit may have a light source and/or a filter unit and/or a coupling unit that is configured to generate the visually distinguishable property of the angle regions and/or of the boundary line.

In one embodiment, the light guidance unit may have a light source that is configured to generate the visually distinguishable property of the at least two angle regions and/or of the boundary line. For this purpose, the light source may be configured, for example, to emit (e.g., to generate) a light fan beam in each angle region and/or the boundary line having the visually distinguishable property.

Alternatively or additionally, the light guidance unit may have a filter unit (e.g., an optical filter, such as a filter foil) that is configured for generating the visually distinguishable property of the at least two angle regions and/or of the boundary line. Therein, the filter unit may be arranged in a ray path of the light source for emitting the at least one light fan beam.

Alternatively or additionally, the light guidance unit may have a coupling unit that is configured for generating the visually distinguishable property of the at least two angle regions and/or of the boundary line. Therein, the coupling unit may be configured to couple the light emitted by at least two (e.g., different) light sources (e.g., using a partially transmissive optical beam-splitter and/or a beam-splitter cube and/or a partially silvered mirror). Therein, the visually distinguishable property of the at least two angle regions and/or of the boundary line may be capable of being generated by the light from the at least two light sources coupled together by the coupling unit.

The embodiment may enable a generation of the visually distinguishable property of the angle regions and/or of the boundary line via the light guidance unit.

In a further embodiment of the apparatus, the light guidance unit may be configured to adapt the angle regions dependent upon the planning information.

The light guidance unit may be configured to adapt a spatial positioning (e.g., a spatial orientation) of the at least two angle regions (e.g., the overall angle region) dependent upon the planning information. Further, the light guidance unit may be configured to adapt each angle of the at least two angle regions dependent upon the planning information (e.g., to reduce or increase them). Therein, the light guidance unit may be configured to adapt a spatial positioning (e.g., an orientation) of the boundary line dependent upon the planning information.

The adaptation of the at least two angle regions dependent upon the planning information may take place, for example, by adapting a projection geometry of the light fan beam (e.g., using an aperture and/or a mirror and/or an imaging optical system) and/or by repositioning (e.g., pivoting and/or tilting and/or rotating and/or orienting and/or moving) the light guidance unit (e.g., the light source). Further, the adaptation of the at least two angle regions dependent upon the planning information may take place in a motorized manner (e.g., by motorized adaptation of the projection geometry of the light fan beam, such as of an imaging optical system) and/or by repositioning the light guidance unit.

The embodiment may enable a flexible adaptation of the angle regions (e.g., also a positioning of the boundary line) dependent upon the planning information.

In a further embodiment of the apparatus, the light guidance unit may be configured to emit a first and a further light fan beam dependent upon the planning information. Therein, the first light fan beam may have two different first angle regions that abut one another along a first boundary line. Further, the first angle regions and/or the first boundary line may have a visually distinguishable property. In addition, an intersection point of the first boundary line with the further light fan beam, in the operating state of the apparatus, may illuminate at least a part of the planned positioning of the medical object.

According to a first variant, the light guidance unit may have two light guidance sub-units, each having a light source. Therein, the light guidance sub-units (e.g., each light source) may be configured for emitting one of the two light fan beams dependent upon the planning information. Therein, a first light guidance sub-unit of the light guidance sub-units may include a light source and/or a filter unit and/or a coupling unit that generates the visually distinguishable property of the first angle regions and/or the first boundary line of the first light fan beam. The light guidance sub-units may be arranged in a defined arrangement (e.g., spaced apart). For example, the coupling unit (e.g., a partially transmissive optical beam-splitter and/or a beam-splitter cube and/or a partially silvered mirror) may be configured to couple the light emitted by the at least two (e.g., different) light sources.

According to a further variant, the light guidance unit (e.g., the light source) may be configured for emitting the two light fan beams dependent upon the planning information. Therein, the light guidance unit may include the light source and/or a filter unit that generate the visually distinguishable property of the first angle regions and/or the first boundary line of the first light fan beam.

The visually distinguishable property of the first angle regions and/or of the first boundary line may include at least one of the following features: a color, a spatial and/or temporal pattern, a polarization, a brightness, a line thickness, and/or an unsharpness. If the at least two angle regions have a different color, then the first boundary line may be formed by a color transition between the two first angle regions.

In one embodiment, the two light fan beams may be arranged angled (e.g., perpendicularly) to one another and intersect along a common beam. Thus, the two light fan beams may project two mutually crossing lines. Further, the first boundary line may have a point of intersection with the further light fan beam (e.g., a line projected via the further light fan beam). Therein, the intersection point of the first boundary line with the further light fan beam may illuminate at least a part of the planned positioning of the medical object.

The embodiment may enable a more precise positioning of the medical object.

In a further embodiment of the apparatus, the further light fan beam may have two different further angle regions that abut one another along a further boundary line. Therein, the first and the further angle regions and/or the first and further boundary line may have a visually distinguishable property. In addition, an intersection point of the first and the further boundary line in the operating state of the apparatus may illuminate at least a part of the planned positioning of the medical object.

According to a first variant, the light guidance unit may have two light guidance sub-units, each having a light source. Therein, the light guidance sub-units (e.g., each light source) may be configured for emitting one of the two light fan beams, dependent upon the planning information. Therein, a first light guidance sub-unit of the light guidance sub-units may include a light source and/or a filter unit that generates the visually distinguishable property of the first angle regions and/or of the first boundary line of the first light fan beam. Further, a further light guidance sub-unit of the light guidance sub-units may include a light source and/or a filter unit that generates the visually distinguishable property of the further angle regions and/or the further boundary line of the first light fan beam. The light guidance sub-units may be arranged in a defined arrangement (e.g., spaced apart).

According to a further variant, the light guidance unit (e.g., the light source) may be configured for emitting the two light fan beams dependent upon the planning information. Therein, the light guidance unit may include the light source and/or a filter unit and/or a coupling unit that generate the visually distinguishable property of the first and further angle regions and/or the first and the further boundary line of each light fan beam.

The visually distinguishable property of the first and further angle regions and/or of the first and further boundary line may include at least one of the following features: a color, a spatial and/or temporal pattern, a polarization, a brightness, a line thickness, and/or an unsharpness. If the at least two further angle regions have a different color, then the boundary line may be formed by a color transition between the two further angle regions.

In one embodiment, the two light fan beams may be arranged angled (e.g., perpendicularly) to one another and intersect along a common beam. Thus, the two light fan beams may project two mutually crossing lines. Further, the first boundary line may have a point of intersection with the further boundary line. Therein, the intersection point of the first boundary line with the further boundary line may illuminate at least a part of the planned positioning of the medical object (e.g., a planned insertion point of the medical object into the examination object). For the illumination (e.g., marking) of the planned insertion point, the light guidance unit does not necessarily have to be arranged in the bull's-eye view in relation to a planned trajectory for arranging the medical object. Rather, another positioning may be selected, where a planned spatial position (e.g., a planned 3D location) of the planned insertion point is to be known. Whether the lines projected by the two light fan beams at the planned insertion point illuminate a surface (e.g., a skin surface of the examination object) may be visually checked by the medical operating person by checking whether the two boundary lines have a common intersection point. By this, registration errors may be identified.

The embodiment may enable a more precise positioning of the medical object.

In a further embodiment of the apparatus, the medical object may have at least one marker structure. In the operational state of the apparatus, the light fan beams and/or the boundary line may illuminate the marker structure exactly when the medical object is arranged according to the planned positioning.

The light fan beams and/or the boundary line may represent a predefined light distribution that projects a predefined light pattern. The marker structure may be configured as a structural element (e.g., as a cut-out and/or elevation and/or edge and/or recognizable symmetry, such as on a surface of the medical object). Alternatively or additionally, the marker structure may be configured as a graphical element (e.g., as a pattern and/or line and/or stripe and or cross) on the surface of the medical object. Alternatively or additionally, the marker structure may be configured as a light-reflecting element (e.g., as a reflector) and/or as a light-absorbing element (e.g., a black, such as deep black coating on the surface of the medical object). Therein, the light-reflecting element may be configured for at least partial (e.g., complete) reflection of the light distribution (e.g., of the at least one light fan beam and/or the boundary line). Further, the light-absorbing element may be configured for at least partial (e.g., complete) absorption of the light distribution (e.g., of the at least one light fan beam and/or the boundary line). In one embodiment, the light-reflecting element may have a reflectivity that is at least partially different from that of the surface of the medical object. Further, the light-absorbing element may have an absorptivity that is at least partially different from that of the surface of the medical object. The marker structure may be arranged, for example, on a shaft of the medical object.

In one embodiment, in an operating state of the apparatus, the light guidance unit may emit the at least one light fan beam, such that the light fan beam and/or the boundary line may illuminate the marker structure of the medical object exactly when the medical object is arranged according to the planned positioning.

The embodiment may enable a particularly reliable positioning of the medical object.

In a further embodiment of the apparatus, the planning information may specify the planned positioning of the medical object in relation to an examination object. The apparatus may include a sensor for detecting the momentary positioning of the examination object. The control unit may be configured to register the planning information with the momentary positioning of the examination object.

The sensor may be configured, for example, as an optical, electromagnetic, and/or acoustic sensor. The optical sensor may detect a momentary positioning of the examination object using light. The optical sensor may include, for example, a 2D or 3D camera (e.g., a mono or stereo camera). The acoustic sensor may detect, for example, ultrasonic waves that have been reflected after striking the examination object. From a transit time of the ultrasonic waves from a transmission of the ultrasonic waves until the detection of the ultrasonic waves, the momentary positioning of the examination object may be detected. The electromagnetic sensor may detect the momentary positioning of the examination object using magnetic fields (e.g., magnetic field changes). For example, the electromagnetic sensor may be configured to detect the momentary positioning of the examination object based on a change in an electrical capacitance via the examination object.

Alternatively or additionally, the sensor may be configured as a medical imaging device. Therein, the momentary positioning of the examination object may be detected based on image data recorded by the medical imaging device. The medical imaging device may include, for example, a magnetic resonance tomography (MRT) system and/or a computed tomography (CT) system and/or a medical X-ray device (e.g., a medical C-arm X-ray device) and/or an ultrasound device and/or a positron emission tomography (PET) system.

The registration of the planning information with the momentary positioning of the examination object may include an application of a transformation rule to the planning information. Therein, the transformation rule may specify a transformation (e.g., a translation and/or rotation and/or deformation and/or scaling) that minimizes a deviation between a coordinate system of the planning information and a coordinate system of the momentary positioning of the examination object (e.g., a coordinate system of the examination object).

The embodiment may enable an improved positioning of the medical object in relation to the examination object.

In a further embodiment of the apparatus, the apparatus may also have a medical imaging device. Therein, the light guidance unit may be arranged on the medical imaging device and/or may be integrated at least partially into the medical imaging device.

The medical imaging device may include a medical X-ray device (e.g., a medical C-arm X-ray device and/or a cone beam computed tomography (CBCT) system) and/or a computed tomography (CT) system and/or a magnetic resonance tomography (MR) system and/or a positron emission tomography (PET) system and/or an ultrasound device. In one embodiment, the imaging device may be configured for recording and providing image data from the examination object (e.g., from the examination object and the medical object).

In one embodiment, the light guidance unit may be arranged on the medical imaging device (e.g., a source or a detector), such as fastened onto and/or at least partially (e.g., entirely) integrated into the medical imaging device.

By this, an inherent registration may exist between a coordinate system of the light guidance unit and a coordinate system of the imaging device, whereby an improved positioning of the medical object may be enabled.

In a further embodiment of the apparatus, the medical imaging device may be configured as a medical X-ray device for recording X-ray image data from an examination object. The X-ray device may include an X-ray detector and an X-ray source that are arranged opposite one another and in a defined arrangement. The light guidance unit may be arranged on the X-ray detector, the X-ray source, or a common holding structure of the defined arrangement.

The X-ray source may be configured to emit X-ray radiation (e.g., an X-ray bundle). Further, the X-ray detector may be configured for acquiring (e.g., detecting) incident X-ray radiation (e.g., on an X-ray-sensitive surface). The X-ray source and the X-ray detector may be arranged opposite one another. For example, the X-ray source and the X-ray detector may be arranged opposite one another such that the X-ray radiation that may be emitted from the X-ray source strikes the X-ray detector (e.g., the X-ray-sensitive surface of the X-ray detector). Further, the X-ray source and the X-ray detector may be arranged on a common holding structure (e.g., a C-arm and/or an O-arm) in a defined arrangement relative to one another. The defined arrangement of the X-ray source and the X-ray detector (e.g., the common holding structure) may be mounted to be movable (e.g., translatable and/or rotatable).

The light guidance unit may be arranged on the X-ray detector, the X-ray source, or a common holding structure of the defined arrangement (e.g., fastened onto and/or at least partially, such as completely, integrated into the same).

The embodiment may enable an improved positioning of the medical object under X-ray imaging control.

The present embodiments relate in a second aspect to a method for emitting a light distribution. In a first act, a planning information item regarding a planned positioning of the medical object is provided. In a further act, a light fan beam is emitted, dependent upon the planning information item, by a light guidance unit. The light fan beam has two different angle regions. Therein, the angle regions abut one another along a boundary line. The angle regions and/or the boundary line have a visually distinguishable property. The boundary line illuminates at least a part of the planned positioning of the medical object.

The advantages of the method of the present embodiments substantially correspond to the advantages of the apparatus of the present embodiments for positioning a medical object. Features, advantages, or alternative embodiments mentioned herein may also be transferred to the other claimed subject matter and vice versa.

In a further embodiment of the method, the visually distinguishable property of the angle regions and/or of the boundary line may include at least one of the following features: a color, a spatial and/or temporal pattern, a polarization, a brightness, a line thickness, and/or an unsharpness.

In a further embodiment of the method, the angle regions may be adapted dependent upon the planning information.

In a further embodiment of the method, a first and a further light fan beam may be emitted dependent upon the planning information. Therein, the first light fan beam may have two different first angle regions that abut one another along a first boundary line. Further, the first angle regions and/or the first boundary line may have a visually distinguishable property. Therein, an intersection point of the first boundary line with the further light fan beam may illuminate at least a part of the planned positioning of the medical object.

In a further embodiment of the method, the further light fan beam may have two different further angle regions that abut one another along a further boundary line. Further, the first and the further angle regions and/or the first and the further boundary line may have a visually distinguishable property. Therein, an intersection point of the first and the further boundary line may illuminate at least a part of the planned positioning of the medical object.

In a further embodiment of the method, the medical object may have at least one marker structure. The light fan beam and/or the boundary line may illuminate the marker structure exactly when the medical object is arranged according to the planned positioning.

In a further embodiment of the method, the planning information may specify the planned positioning of the medical object in relation to an examination object. The apparatus may include a sensor for detecting a momentary positioning of the examination object. The planning information may be registered with the momentary positioning of the examination object.

The present embodiments relate, in a third aspect, to a computer program product including a computer program that may be loaded directly into the memory store of a control unit. The computer program includes program portions in order to carry out all the acts of a method of the present embodiments for positioning a medical object when the program portions are executed by the control unit.

The computer program product may include, for example, an item of software with a source code that is still to be compiled and linked or which is only to be interpreted, or an executable software code that, for execution, is still to be loaded into the control unit. Using the computer program product, the method for positioning a medical object by a control unit may be carried out rapidly, exactly reproducibly, and robustly. The computer program product is configured so that the computer program product may carry out the method acts according to the present embodiments using the control unit.

The computer program product is stored, for example, on a computer-readable storage medium or is deposited on a network or server from where the computer program product may be loaded into the processor of a control unit that may be configured directly connected to the control unit or as part of the control unit. Further, control information of the computer program product may be stored on an electronically readable data carrier. The items of control information of the electronically readable data carrier may be configured such that the items of control information carry out a method according to the present embodiments when the data carrier is used in a control unit. Examples of electronically readable data carriers are a DVD, a magnetic tape, or a USB stick, on which electronically readable control information (e.g., software) is stored. If this control information is read from the data carrier and stored in a control unit, all the embodiments of the above-described methods may be carried out.

A realization largely through software has the advantage that conventionally used control units may also easily be upgraded with a software update in order to operate in the manner according to the present embodiments. Where relevant, such a computer program product may include, in addition to the computer program, further constituents, such as, for example, documentation and/or additional components as well as hardware components(e.g., hardware keys (dongles, etc.)) in order to use the software.

### BRIEF DESCRIPTION OF THE DRAWINGS

Example embodiments of the invention are illustrated in the drawings and are described in greater detail below. In the different figures, the same reference signs are used for the same features. In the drawings:

FIGS. 1, 2, 5, and 11 show schematic illustrations of different embodiments of an apparatus for positioning a medical object;

FIGS. 3, 4, and 6 to 10 show schematic illustrations of different operating states of different embodiments of an apparatus for positioning a medical object;

FIGS. 12 and 13 show schematic illustrations of embodiments of a method for emitting a light distribution.

### DETAILED DESCRIPTION

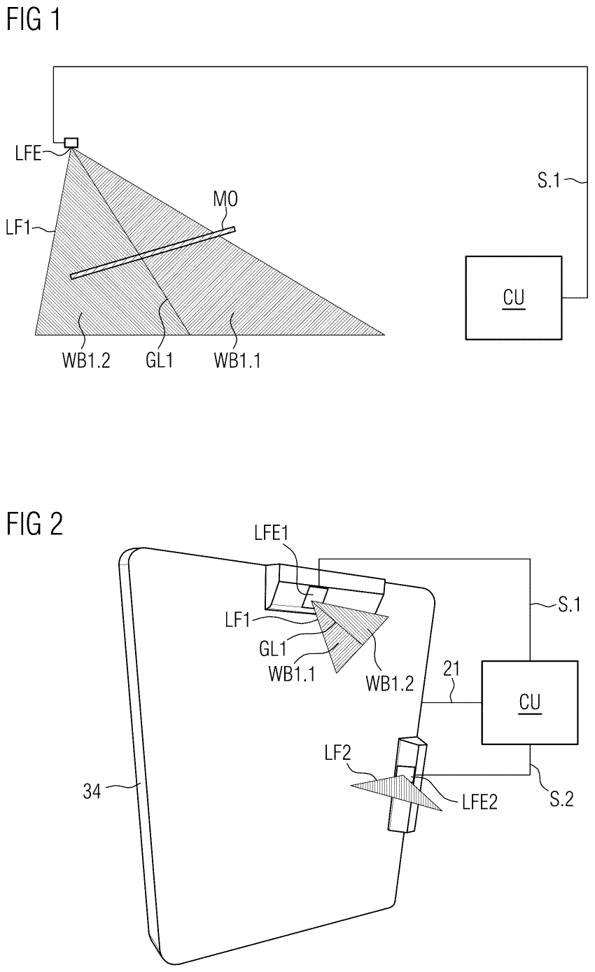

FIG. 1 shows a schematic illustration of an embodiment of an apparatus for positioning a medical object MO. The apparatus may include a control unit CU and a light guidance unit LFE. The control unit CU may be configured to provide a planning information item regarding a planned positioning of the medical object MO. Further, the light guidance unit LFE may be configured to emit at least one light fan beam LF1 dependent upon the planning information. For this purpose, the control unit CU may provide a signal S to the light guidance unit LFE. Therein, the light fan beam LF1 may have two different angle regions WB1.1 and WB1.2 that abut one another along a boundary line GL1. In addition, the angle regions WB1.1 and WB1.2 and/or the boundary line GL1 may have a visually distinguishable property. Therein, the boundary line GL1 illuminates at least a part of the planned positioning of the medical object MO.

In one embodiment, the visually distinguishable property of the angle regions WB1.1 and WB1.2 and/or of the boundary line GL1 may include at least one of the following features: a color, a spatial and/or temporal pattern, a polarization, a brightness, a line thickness, and/or an unsharpness.

In one embodiment, the light guidance unit LFE may have a light source and/or a filter unit for generating the visually distinguishable property of the angle regions WB1.1 and WB1.2 and/or of the boundary line GL1. Further, the light guidance unit LFE may be configured to adapt the angle regions WB1.1 and WB1.2 dependent upon the planning information.

FIG. 2 shows a schematic illustration of a further embodiment of an apparatus for positioning a medical object MO. Therein, the light guidance unit LFE may include two light guidance sub-units LFE1 und LFE2. The two light guidance sub-units LFE1 and LFE2 may be arranged in a defined arrangement and spaced apart from one another (e.g., fastened onto or integrated into an X-ray detector 34 of a medical X-ray device). The X-ray detector 34 may be configured for detecting X-ray radiation incident upon an X-ray-sensitive surface. Further, the X-ray detector 34 may be configured for providing a signal 21 to the control unit CU dependent upon the detected X-ray radiation. The first light guidance sub-unit LFE1 may be configured to emit a first light fan beam LF1 dependent upon the planning information. Further, the further light guidance sub-unit LFE2 may be configured to emit a further light fan beam LFE2 dependent upon the planning information. The first light fan beam LF1 may have two different first angle regions WB1.1 and WB1.2 that abut one another along a first boundary line GL1. In addition, the first angle regions WB1.1 and WB1.2 and/or the first boundary line GL1 may have a visually distinguishable property. Therein, an intersection point of the first boundary line GL1 with the further light fan beam LF2 may illuminate at least a part of the planned positioning of the medical object MO.

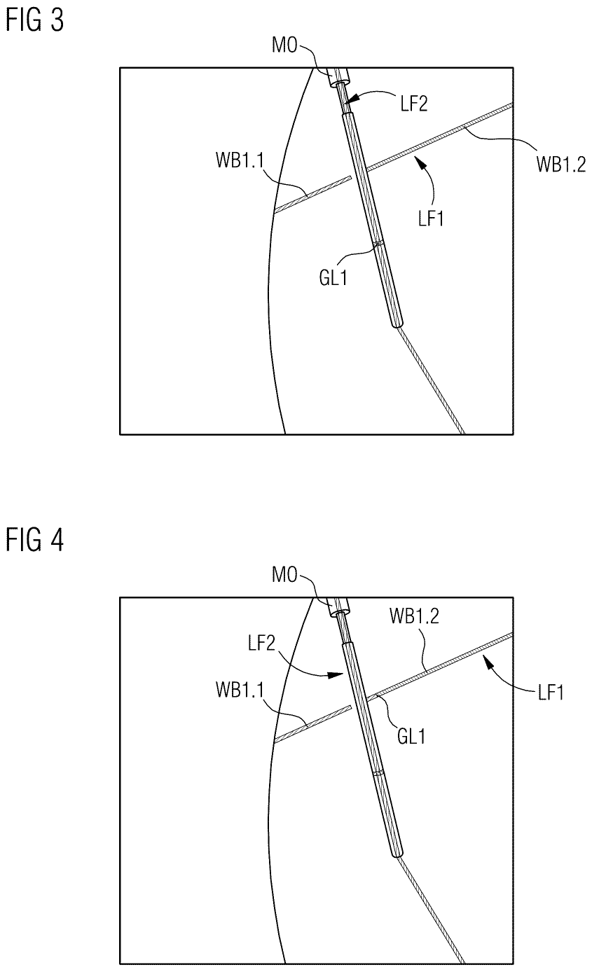

FIGS. 3 and 4 show schematically two different operating states of an embodiment of an apparatus for positioning a medical object MO. In FIG. 3, an arrangement of the medical object MO in accordance with the planned positioning is shown schematically. Therein, the further light fan beam LF2 may illuminate the medical object MO in its planned positioning along its longitudinal extent direction (e.g., laterally in the progression view). Further, the first light fan beam LF1 may illuminate the medical object MO in its planned positioning such that the boundary line GL1 intersects the further light fan beam LF2 on the longitudinal extent direction of the medical object MO (e.g., along a planned path for the arrangement of the medical object).

FIG. 4 shows schematically an arrangement of the medical object MO deviating from the planned positioning. Therein, the further light fan beam LF2 may illuminate the medical object MO along its longitudinal extent direction, although only the first angle region WB1.1 of the first light fan beam LF1 illuminates the medical object MO and/or intersects the further light fan beam LF2 along the longitudinal extent direction of the medical object MO. By this, apart from the deviating arrangement of the medical object MO relative to the planned positioning, an information item regarding the repositioning into the planned positioning may be provided.

FIG. 5 shows a schematic representation of a further embodiment of an apparatus for positioning a medical object MO. Therein, the further light fan beam LF2 may have two different further angle regions WB2.1 and WB2.2 that abut one another along a further boundary line GL2. Further, the first and the further angle regions WB1.1, WB1.2, WB2.1 and WB2.2 and/or the first and the further boundary line GL1 and GL2 may have a visually distinguishable property. In addition, an intersection point of the first and the further boundary line GL1 and GL2 may illuminate at least a part of the planned positioning of the medical object MO.

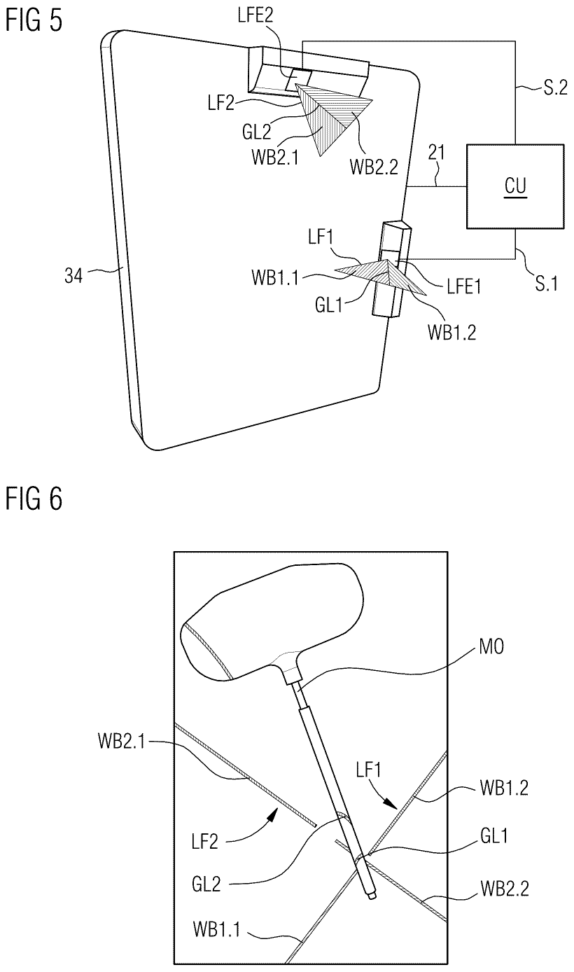

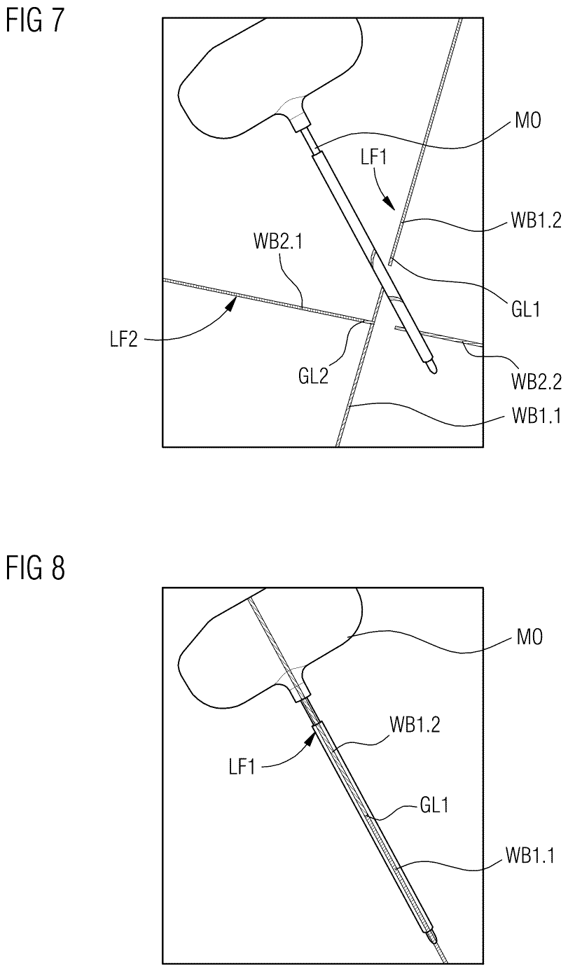

FIGS. 6 and 7 show schematically two different operating states of an embodiment of an apparatus for positioning a medical object MO. Therein, the medical object MO may be configured, for example, as a trocar. Therein, the first and the second light fan beams LF1 and LF2 may each project lines. The lines may have portions corresponding to the first and further angle regions WB1.1, WB1.2, WB2.1 and WB2.2 that have the visually distinguishable property (e.g., a different light color).

In FIG. 6, an arrangement of the medical object MO in accordance with the planned positioning is shown schematically. Therein, the first light fan beam LF1 may illuminate the medical object MO in its planned positioning such that the first boundary line GL1 illuminates the medical object MO (e.g., along its longitudinal extent direction). Further, the further light fan beam LF2 may illuminate the medical object MO in its planned positioning, such that the further boundary line GL2 illuminates the medical object MO (e.g., along its longitudinal extent direction).

FIG. 7 shows schematically an arrangement of the medical object MO deviating from the planned positioning. Therein, the first and the further light fan beam LF1 and LF2 may illuminate the medical object MO, although only the first angle region WB1.1 of the first light fan beam LF1 and the further angle region WB2.2 of the further light fan beam LF2 illuminate the medical object MO. Based on the visually distinguishable property of the first and further angle regions WB1.1 and WB2.2 that illuminate the medical object, information regarding the repositioning into the planned positioning may be provided. In the operating state of the apparatus as shown, a multicolored line projected by each of the first and further light fan beams LF1 and LF2 cannot be oriented along the planned trajectory for arranging the medical object MO (e.g., a planned needle trajectory). The slice illuminated by the respective light fan beams LF1 or LF2 (e.g., the approximately illuminated plane) intersects the planned needle trajectory, at least in the operating state shown, with a spatial angle of at least 20° or at least 40° (e.g., between 60° and 90°). An emission direction of the respective boundary line GL1 and GL2 may be set such that the emission direction extends through a spatial intersection point between the associated light fan beam and the needle trajectory. By this, information regarding the repositioning into the planned positioning (e.g., a guidance and/or orientation information item) may be provided (e.g., to a medical operating person).

The two-colored light projected by the first and further light fan beams LF1 and LF2 (e.g., laser lines) may each show a color transition line as the boundary line GL1 and GL2 in space. The guidance information to the medical operating person may consist of arranging the instrument such that both boundary lines GL1 and GL2 illuminate (e.g., intersect) the medical object in a defined manner so that on a surface of the medical object, a transition point is visible at a predefined position. In one embodiment, a part of the needle trajectory may be already specified in advance (e.g., via an indication of an insertion point, such as a skin incision point, on a surface of the examination object at an earlier time point). Alternatively or additionally, the first and the second light fan beams each project the respective line onto an alternative surface (e.g., a surface of a white sterile glove of the medical operating person when the operating person grips the medical object close to the illuminated site).

FIG. 8 shows schematically a further operating state of an embodiment of an apparatus for positioning a medical object MO. Therein, the medical object MO may have at least one marker structure MK. Therein, the first boundary line GL1 may illuminate the marker structure MK exactly when the medical object MO is arranged according to the planned positioning (e.g., in a target depth in relation to the examination object). Further, the first angle regions WB1.1 and WB1.2 of the first light fan beam LF1 may illuminate the medical object MO along its longitudinal extent direction if the medical object MO is arranged according to its planned positioning.

In the example embodiment shown in FIG. 8, the planned trajectory (e.g., a needle trajectory) may be established for arranging the medical object MO within the slice (e.g., plane) that is illuminated by the first light fan beam LF1. The first boundary line GL1 may be capable of being displaced by adapting the angle regions WB1.1 and WB1.2 of the first light fan beam LF1 (e.g., along the planned trajectory). The first boundary line may therein provide an information item (e.g., the target depth and/or an advancing speed of the medical object).

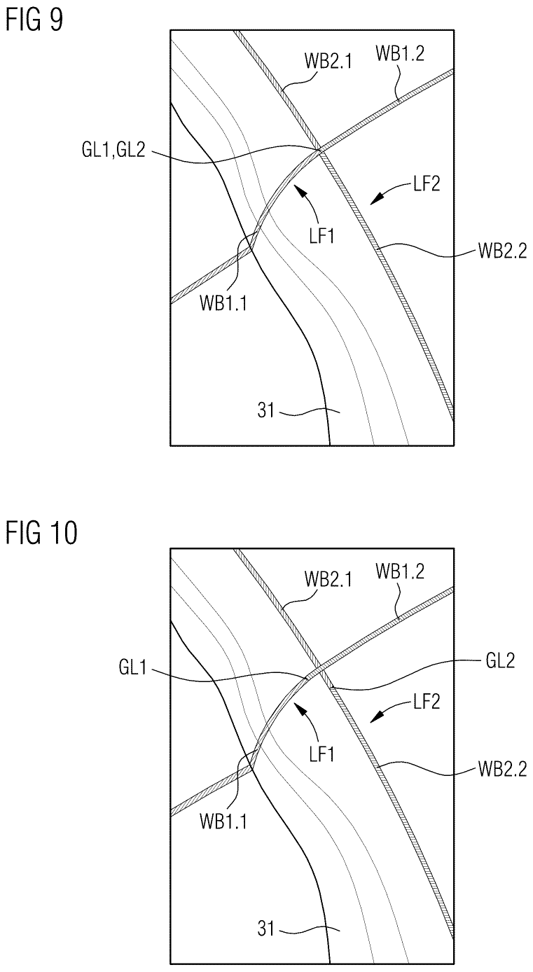

FIGS. 9 and 10 show schematically two different operating states of an embodiment of an apparatus for positioning a medical object MO. Therein, the intersection point of the first boundary line GL1 with the further boundary line GL2 may illuminate a planned insertion point IP (e.g., a puncture site) as part of the planned positioning of the medical object MO on a surface (e.g., a skin surface or a phantom surface) of the examination object 31. The planning information regarding the planned insertion point IP may have been determined (e.g., manually or automatically) based on an image dataset (e.g., pre-procedural image dataset) of the examination object 31. Therein, FIG. 9 shows schematically an operating state of the apparatus, where the planning information (e.g., the light guidance unit LFE) and the momentary positioning of the examination object 31 are correctly registered to one another. Further, FIG. 10 shows schematically an operating state of the apparatus, where the planning information (e.g., the light guidance unit LFE) and the momentary positioning of the examination object 31 are not correctly registered to one another. Therein, the first and the further boundary line GL1 and GL2 are not arranged at a common point.

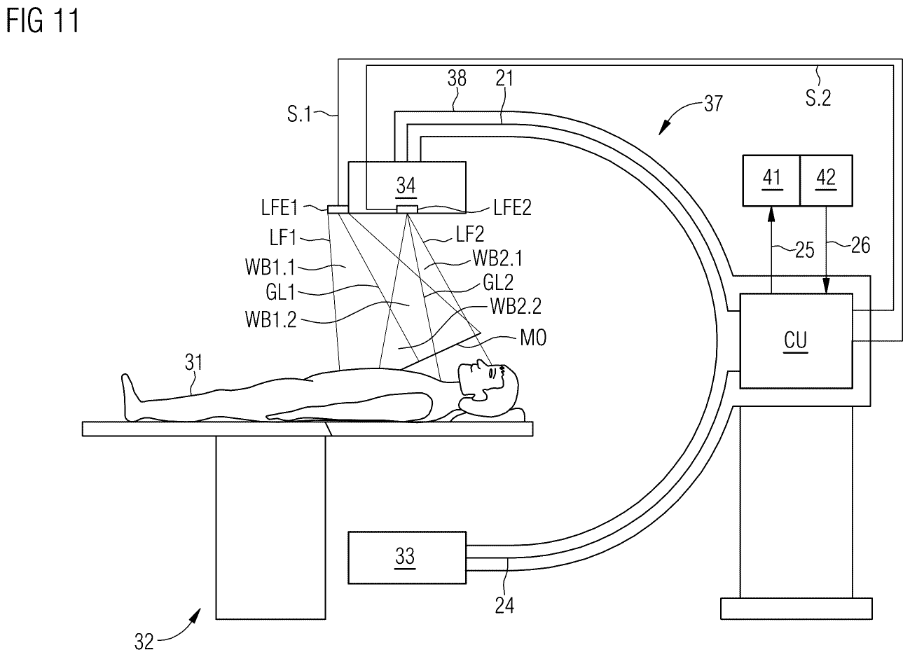

FIG. 11 shows a schematic illustration of a further embodiment of an apparatus for positioning a medical object MO. Therein, the apparatus may include a medical X-ray device 37 (e.g., a medical C-arm X-ray device) for recording X-ray image data of an examination object 31. The X-ray device 37 may include an X-ray source 33 and an X-ray detector 34 that are arranged opposite one another and in a defined arrangement on a C-arm 38. The C-arm 38 may be mounted movably about one or a number of (e.g., several) axes. The control unit CU may transmit a signal 24 to the X-ray source 33. Thereupon, dependent upon the signal 24, the X-ray source 33 may emit X-ray radiation to irradiate (e.g., to transirradiate) the examination object 31 positioned on a patient positioning apparatus 32. When the X-ray beam is incident upon an X-ray-sensitive surface of the X-ray detector 34 after an interaction with the examination object 31, the X-ray detector 34 may emit a signal 21 to the control unit CU. The control unit CU may be configured to detect X-ray image data based on the signal 21.

In one embodiment, the light guidance unit LFE may be arranged on the X-ray detector 34. In one embodiment, the planning information may specify the planned positioning of the medical object MO in relation to the examination object 31. The X-ray device 37 may be configured for detecting a momentary positioning of the examination object 31. Further, the control unit CU may be configured to register the planning information with the momentary positioning of the examination object 31.

The X-ray device may further have an input unit 42 (e.g., a keyboard and/or a joystick) and a display unit 41 (e.g., a monitor and/or a display and/or a projector). The input unit 42 may be integrated into the display unit 41 (e.g., in the case of a capacitive and/or resistive input display). The display unit 41 may be configured to display a graphical representation of X-ray image data recorded using the X-ray device. For this purpose, the control unit CU may transmit a signal 25 to the display unit 41. Further, the input unit may be configured to detect a user input. The detecting unit 42 may further be configured to provide a signal 26 to the control unit CU dependent upon the detected user input. The control unit CU may be configured to control the light guidance unit LFE and/or the X-ray device 37 dependent upon the user input (e.g., the signal 26).

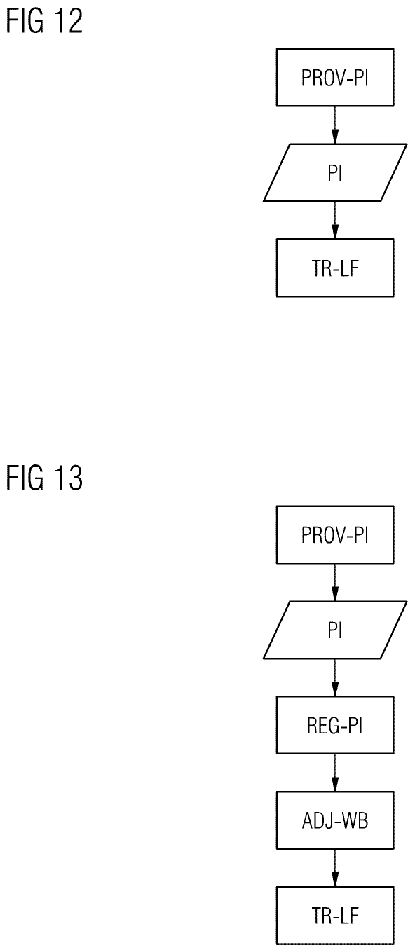

FIG. 12 shows a representation of an embodiment of a method for emitting a light distribution. In a first act, a planning information item PI regarding a planned positioning of the medical object MO is provided PROV-PI. In a further act, at least one light fan beam may be emitted TR-LF by a light guidance unit LFE, dependent upon the planning information item PI. Therein, the at least one light fan beam may have two different angle regions that abut one another along a boundary line. Further, the angle regions and/or the boundary line may have a visually distinguishable property. In addition, the boundary line may illuminate at least a part of the planned positioning of the medical object MO.

In one embodiment, the visually distinguishable property of the angle regions and/or of the at least one boundary line may include at least one of the following features: a color, a spatial and/or temporal pattern, a polarization, a brightness, a line thickness, and/or an unsharpness.

FIG. 13 shows a representation of a further embodiment of a method for emitting a light distribution. Therein, the angle regions may be adapted ADJ-WB dependent upon the planning information PI. Further, a first and a further light fan beam may be emitted TR-LF dependent upon the planning information PI. Therein, the first light fan beam LF1 may have two different first angle regions WB1.1 and WB1.2 that abut one another along a first boundary line GL1. In addition, the first angle regions WB1.1 and WB1.2 and/or the first boundary line GL1 may have a visually distinguishable property. Further, an intersection point of the first boundary line GL1 with the further light fan beam LF2 may illuminate at least a part of the planned positioning of the medical object MO. In one embodiment, the further light fan beam LF2 may have two different further angle regions WB2.1 and WB2.2 that abut one another along a further boundary line GL2. Further, the first and the further angle regions WB1.1, WB1.2, WB2.1 and WB2.2 and/or the first and the further boundary line GL1 and GL2 may have a visually distinguishable property. In addition, an intersection point of the first and the further boundary line GL1 and GL2 may illuminate at least a part of the planned positioning of the medical object MO.

Further, the medical object MO may have at least one marker structure MK. Therein, the at least one light fan beam and/or the boundary line may illuminate the marker structure MK exactly when the medical object MO is arranged according to the planned positioning.

Further, the planning information PI may specify the planned positioning of the medical object MO in relation to an examination object 31. Therein, the apparatus may include a sensor for detecting a momentary positioning of the examination object 31. Further, the planning information PI may be registered REG-PI with the momentary positioning of the examination object 31.

The schematic representations contained in the drawings described do not reveal any scale or size relationships.

The methods described above in detail and the apparatuses disclosed are merely example embodiments that may be modified by a person skilled in the art in a wide variety of ways without departing from the scope of the invention. Further, the use of the indefinite article “a” or “an” does not preclude the possibility that the relevant features may also be present plurally. Similarly, the expressions “unit” and “element” do not preclude the components in question consisting of a plurality of cooperating subcomponents that may possibly also be spatially distributed.

The expression “based upon/on the basis of” may be understood in the context of the present application to have the sense, for example, of the expression “making use of.” In particular, a formulation according to which a first feature is generated (e.g., alternatively, established, determined, etc.) based upon a second feature does not preclude the first feature being able to be generated (e.g., alternatively, established, determined, etc.) based upon a third feature.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. An apparatus for positioning a medical object, the apparatus comprising:
a controller; and
a light guidance unit,
wherein the controller is configured to provide a planning information item regarding a planned positioning of the medical object,
wherein the light guidance unit is configured to emit at least one light fan beam dependent upon the planning information item,
wherein the at least one light fan beam has two different angle regions that abut one another along a boundary line,
wherein the two different angle regions, the boundary line, or the two different angle regions and the boundary line have a visually distinguishable property, and
wherein, in an operating state of the apparatus, the boundary line illuminates at least a part of the planned positioning of the medical object.

2. The apparatus of claim 1, wherein the visually distinguishable property of the two different angle regions, the boundary line, or the two different angle regions and the boundary line comprises:
a color;
a spatial, temporal, or spatial and temporal pattern;
a polarization;
a brightness;
a line thickness;
an unsharpness; or
any combination thereof.

3. The apparatus of claim 1, wherein the light guidance unit comprises a light source, a filter unit, a coupling unit, or any combination thereof that is configured for generating the visually distinguishable property of the two different angle regions, the boundary line, or the two different angle regions and the boundary line.

4. The apparatus of claim 1, wherein the light guidance unit is configured to adapt the two different angle regions dependent upon the planning information item.

5. The apparatus of claim 1, wherein the light guidance unit is configured to emit a first light fan beam and a further light fan beam dependent upon the planning information item,
wherein the first light fan beam has two different first angle regions that abut one another along a first boundary line,
wherein the two different first angle regions, the first boundary line, or the two different first angle regions and the first boundary line have a visually distinguishable property, and
wherein, in the operating state of the apparatus, an intersection point of the first boundary line with the further light fan beam illuminates at least a part of the planned positioning of the medical object.

6. The apparatus of claim 5, wherein the further light fan beam has two different further angle regions that abut one another along a further boundary line,
wherein the two different first angle regions and the two different further angle regions, the first boundary line and the further boundary line, or a combination thereof has a visually distinguishable property, and
wherein, in the operating state of the apparatus, an intersection point of the first boundary line and the further boundary line illuminates at least a part of the planned positioning of the medical object.

7. The apparatus of claim 1, wherein the medical object has at least one marker structure,
wherein, in the operating state of the apparatus, the at least one light fan beam, the boundary line, or the at least one light fan beam and the boundary line illuminate the at least one marker structure exactly when the medical object is arranged according to the planned positioning.

8. The apparatus of claim 1, wherein the planning information item specifies the planned positioning of the medical object in relation to an examination object,
wherein the apparatus further comprises a sensor for detecting a momentary positioning of the examination object, and
wherein the controller is further configured to register the planning information item with the momentary positioning of the examination object.

9. The apparatus of claim 1, further comprising:
a medical imaging device,
wherein the light guidance unit is arranged on the medical imaging device, is integrated at least partially into the medical imaging device, or a combination thereof.

10. The apparatus of claim 1, wherein the medical imaging device is configured as a medical X-ray device for recording X-ray image data from an examination object,
wherein the X-ray device comprises an X-ray detector and an X-ray source that are arranged opposite one another and in a defined arrangement, and
wherein the light guidance unit is arranged on the X-ray detector, the X-ray source, or a common holding structure of the defined arrangement.

11. A method for emitting a light distribution, the method comprising:
providing a planning information item regarding a planned positioning of a medical object;
emitting at least one light fan beam dependent upon the planning information item, using a light guidance unit of an apparatus,
wherein the at least one light fan beam has two different angle regions that abut one another along a boundary line,
wherein the two different angle regions, the boundary line, or the two different angle regions and the boundary line have a visually distinguishable property, and
wherein the boundary line illuminates at least a part of the planned positioning of the medical object.

12. The method of claim 11, wherein the visually distinguishable property of the two different angle regions, the boundary line, or the two different angle regions and the boundary line comprises:
a color;
a spatial, temporal, or spatial and temporal pattern;
a polarization;
a brightness;
a line thickness;
an unsharpness; or
any combination thereof.

13. The method of claim 11, wherein the two different angle regions are adapted dependent upon the planning information item.

14. The method of claim 11, wherein emitting the at least one light fan beam comprises emitting a first light fan beam and a further light fan beam dependent upon the planning information item,
wherein the first light fan beam has two different first angle regions that abut one another along a first boundary line,
wherein the two different first angle regions, the first boundary line, or the two different first angle regions and the first boundary line have a visually distinguishable property, and
wherein an intersection point of the first boundary line with the further light fan beam illuminates at least a part of the planned positioning of the medical object.

15. The method of claim 14, wherein the further light fan beam has two different further angle regions that abut one another along a further boundary line,
wherein the two different first angle regions and the two different further angle regions, the first boundary line and the further boundary line, or a combination thereof has a visually distinguishable property, and
wherein an intersection point of the first boundary line and the further boundary line illuminates at least a part of the planned positioning of the medical object.

16. The method of claim 11, wherein the medical object has at least one marker structure, and
wherein the at least one light fan beam, the boundary line, or the at least one light fan beam and the boundary line illuminate the marker structure exactly when the medical object is arranged according to the planned positioning.

17. The method of claim 11, wherein the planning information item specifies the planned positioning of the medical object in relation to an examination object,
wherein the apparatus comprises a sensor configured to detect a momentary positioning of the examination object, and
wherein the planning information item is registered with the momentary positioning of the examination object.

18. A non-transitory computer-readable storage medium that stores instructions executable by one or more processors to emit a light distribution, the instructions comprising:
providing a planning information item regarding a planned positioning of a medical object;
emitting at least one light fan beam dependent upon the planning information item, using a light guidance unit,
wherein the at least one light fan beam has two different angle regions that abut one another along a boundary line,
wherein the two different angle regions, the boundary line, or the two different angle regions and the boundary line have a visually distinguishable property, and
wherein the boundary line illuminates at least a part of the planned positioning of the medical object.
